(19) United States
(12) Patent Application Publication
(10) Pub. No.: US 2026/0315052 A1
(43) Pub. Date: Oct. 15, 2026

(54) TRANSORBITAL SURGICAL ACCESS SYSTEMS AND METHODS OF USE

(71) Applicant: SPIWay LLC, Carlsbad, CA (US)

(72) Inventors: Eugene Chen, Carlsbad, CA (US); Richard C. Ewers, Carlsbad, CA (US); Stephanie Frimond, Carlsbad, CA (US)

(21) Appl. No.: 19/639,102

(22) Filed: Apr. 3, 2026

Related U.S. Application Data
(60) Provisional application No. 63/785,997, filed on Apr. 9, 2025.

(51) Int. Cl. A61B 17/00

(52) U.S. Cl. CPC A61B 17/00234; A61B 2017/00336

(57) ABSTRACT

A transorbital surgical access system includes a surgical access shield configured to be positioned within a transorbital surgical corridor between an orbital incision and a surgical target. The shield defines an instrument workspace lumen, and a wall of the shield establishes a mechanical protection boundary between the instrument workspace lumen and medial orbital anatomical structures. In representative embodiments, the shield includes first and second wall regions having different radial expandability so that expansion is preferentially directed toward a surgical workspace and away from protected medial orbital anatomical structures. The shield may include a braided scaffold, an elastomeric coating, and an expanded hourglass profile having a proximal cone and a narrower midsection or waist. Preferential expansion is further achieved by greater medial wall thickness, a specialized medial side coating, or higher medial filament density. Representative deployment features include a confinement housing, a stylet, a guide interface, and an everting deployment structure including a hub and non-mesh sleeve.

### CROSS-REFERENCE TO RELATED APPLICATIONS

This application claims priority to and the benefit of U.S. Provisional Patent Application Ser. No. 63/750,001, filed on Apr. 9, 2025, the entire disclosure of which is incorporated herein by reference.

### TECHNICAL FIELD

The present disclosure relates generally to surgical access devices and methods for transorbital procedures. More particularly, the disclosure relates to surgical access shields and associated delivery systems configured to create a protected instrument workspace within a transorbital surgical corridor.

### BACKGROUND

Transorbital surgery has emerged as a minimally invasive approach for accessing anatomical regions of the skull base and orbit, including the orbital apex, anterior cranial fossa, middle cranial fossa, and infratemporal fossa. Compared with traditional transcranial or endonasal approaches, transorbital approaches may provide a more direct route in selected cases, reduce collateral tissue disruption, shorten the working distance to deep-seated targets, and improve cosmetic outcomes using concealed or otherwise favorable incisions. Advancements in endoscopic and microsurgical techniques have expanded the role of transorbital surgery in treating orbital tumors, skull base lesions, inflammatory conditions, and neurovascular disorders, thereby enabling access to deep-seated lesions with potentially lower morbidity in appropriately selected cases.

Despite those advantages, transorbital endoscopic and microsurgical procedures are performed in a confined space adjacent to delicate neurovascular and soft-tissue structures. Structures that may be vulnerable during corridor creation, retraction, instrument exchange, or operative manipulation include the optic nerve, the ophthalmic artery and its branches, extraocular muscles, the muscle cone, sensory nerves, orbital fat, and components of the lacrimal system. Injury to such structures may result in visual impairment, hemorrhage, diplopia, ptosis, enophthalmos, chronic dry eye, or other significant morbidity. During access, bone removal and retraction may further increase the risk of collateral injury to the frontal branch of the facial nerve or the zygomaticofacial nerve.

One commonly used transorbital approach employs a superior eyelid crease incision to provide access to the lateral orbit and adjacent skull-base anatomy. Although effective in selected cases, that approach may provide only a limited working corridor, and the orbital soft tissues and muscle cone may remain insufficiently protected along the pathway used by the endoscope and associated instruments. Retraction and instrument passage along that pathway may contribute to soft-tissue trauma and prolonged recovery. Conventional retractors and protective sheaths may provide localized tissue displacement or general soft-tissue shielding, but they do not necessarily create a dedicated instrument workspace lumen while simultaneously establishing an anatomically directional protection boundary that preferentially protects vulnerable medial orbital anatomical structures. Existing surgical shields and retractors, including those described in U.S. Pat. No. 9,011,326 B2, may provide basic soft-tissue protection but may lack adaptability to patient-specific anatomy, may fail to create a dedicated instrument workspace lumen, and may not establish a directional protection boundary that preferentially safeguards sensitive medial orbital anatomical structures while permitting controlled expansion toward the surgical workspace. Similarly, braided access sheaths developed for transnasal procedures do not address the asymmetric anatomical constraints of the orbit or the requirement for corridor-specific medial protection.

A need therefore exists for an improved transorbital access system that can be introduced in a low-profile configuration, expanded in a controlled manner within the orbit, and maintained in position to define a protected instrument workspace. A further need exists for a system that can preferentially limit expansion toward medial orbital anatomical structures while permitting greater controlled expansion toward the surgical workspace, and that can be delivered using deployment mechanisms compatible with transorbital operative technique.

### SUMMARY

The present disclosure provides a surgical access system for transorbital surgery. The system includes a surgical access shield, illustrated in many embodiments as shield (5), configured to be positioned within a transorbital surgical corridor extending between an orbital incision (85) and a surgical target. The shield (5) defines an instrument workspace lumen (or lumenal through-path) (26), and a wall (24) of the shield (5) is configured, when the shield (5) is positioned within the corridor, to establish a mechanical protection boundary between the lumen (26) and medial orbital anatomical structures.

In some embodiments, the wall (24) of the shield (5) includes a first wall region having a lower radial expandability than a second wall region. The first wall region is configured for placement adjacent protected medial orbital anatomical structures, while the second wall region is configured to permit greater outward displacement toward the lateral or otherwise less vulnerable side of the operative workspace. In this manner, the shield (5) can expand preferentially toward the surgical workspace while maintaining a more protective medial boundary.

In additional embodiments, preferential radial expansion is achieved by providing the medial side of the wall with a greater thickness than the lateral side, by applying a specialized medial side coating that may include binding agents or therapeutic compositions, or by configuring the medial side of the braided scaffold with a higher filament density than the lateral side. These structural variations further limit expansion adjacent to protected medial orbital anatomical structures while permitting greater expansion laterally, thereby enhancing the mechanical protection boundary.

In various embodiments, the shield (5) may be formed as a braided tubular scaffold or tubular braid (22), a laser-cut or otherwise fenestrated stent-like body, or another self-expanding or resilient tubular structure. The braided embodiment may include interwoven metallic filaments, polymeric filaments, or composite filaments arranged in a helical braid pattern. A coating or sleeving layer, such as silicone or polyurethane, may partially or completely span the interstitial spaces of the braid (22) to form a substantially fluid-impermeable barrier and to tailor compliance in selected regions of the wall (24).

In certain embodiments, the shield (5) assumes an expanded hourglass profile that includes a distal section (10), a narrower mid-section or waist (15), and a larger proximal section (20) that defines a proximal cone (30) or funnel. The distal section (10) may define a distal cone (28). The proximal cone (30) may remain at or adjacent the incision (85) and may protect tissue at the entry site, including adjacent ocular or peri-incisional tissue, while also assisting instrument entry and reducing device migration.

Deployment may be accomplished in several ways. Representative embodiments include a confinement-housing embodiment in which the shield (5) is constrained in a low-profile state within a confinement housing (32) and released by plunger (34) advancement; a stylet-based embodiment in which the shield (5) is axially elongated over a stylet (45) or delivery stylet (75) and then released to expand; a guide-assisted embodiment in which a protection guide (90) having a receiver slot (95) cooperates with a receiver slot interface (100) on the shield (5); and an everting embodiment in which a proximal portion progressively everts and advances distally, optionally by traction applied to a hub (105), to deploy the shield and optionally perform blunt dissection.

The disclosed system may be used in a variety of transorbital procedures, including optic nerve decompression, lesion access, tumor resection, and vascular or skull-base procedures. By maintaining the wall (24) of the shield (5) between operative instruments and medial orbital anatomical structures during use, the system can reduce inadvertent trauma and improve operative control.

The foregoing general description and the following detailed description are merely exemplary. The embodiments described in this summary are illustrative examples and are not intended to limit the scope of the invention, which is defined solely by the claims. Various modifications, alternatives, and additional or different embodiments may be made without departing from the scope of the claims.

### BRIEF DESCRIPTION OF THE DRAWINGS

The accompanying drawings, which are incorporated in and constitute part of this specification, illustrate example embodiments and together with the description, explain various principles of the disclosed embodiments. For clarity, simplicity, and flexibility, not all elements, components, or specifications are defined in all drawings. Not all drawings corresponding to specific steps or example embodiments are drawn to scale. Emphasis is instead placed on illustrating the nature, function, and operation of the disclosed systems and methods.

FIG. 1 depicts a transorbital surgical corridor lateral to the orbital muscle cone, with arrows identifying a medial region in which protective separation from operative instruments is desired.

FIG. 2 depicts a representative transorbital surgical corridor to a lateral orbital or skull-base target, again illustrating the medial region for which protective shielding is desired.

FIG. 3A depicts a side profile view of a shield (5) having an hourglass shape, including distal section (10), mid-section (15), proximal section (20), tubular braid (22), wall (24), distal cone (28), and proximal cone (30).

FIG. 3B depicts an isometric view of the shield (5) of FIG. 3A and further illustrates the instrument workspace lumen or lumenal through-path (26).

FIG. 4 depicts an isometric view of the shield (5) in a deployed hourglass configuration.

FIGS. 5A and 5B depict the shield (5) in a drawn-down low-profile insertion or delivery state.

FIGS. 6A and 6B depict an alternate embodiment in which the proximal cone (30) is deployed and a cylindrical or reduced-profile section may spontaneously expand, with FIG. 6B further depicting an embodiment without a distal cone (28).

FIG. 7 schematically depicts the shield (5) divided into distal section (10), midsection (15), and proximal section (20), and illustrates representative functions of those sections.

FIGS. 8A-8C depict a deployment system utilizing a confinement housing (32) and plunger (34), with plunger deployment direction (35) and plunger removal direction (40).

FIGS. 9A-9C depict a stylet-based deployment system utilizing stylet (45), distal confinement structure (50), proximal confinement structure (55), stylet deployment direction (60), stylet retraction direction (65), and lumen (26).

FIGS. 10A and 10B depict a shield (5) having a distal eyelet or stylet receiver (70), a delivery stylet (75), and a distal stop (80).

FIGS. 11A and 11B depict an embodiment of the shield (5) without a drawn-down waist-like mid-section (15).

FIG. 12 depicts non-limiting cross-sectional profiles for the shield (5), including circular, elliptical, lobed, bean-shaped, and tear-drop profiles.

FIGS. 13A and 13B depict an incision (85) and a protection guide (90) configured for insertion through the incision and along the surgical pathway.

FIGS. 14A and 14B depict the protection guide (90) having a receiver slot (95).

FIG. 15 depicts a shield (5) having a receiver slot interface (100), together with stylet (45) and distal eyelet or stylet receiver (70).

FIG. 16 depicts the shield (5) and stylet (45) being advanced along protection guide (90) with the receiver slot interface engaged in receiver slot (95) and with advancement occurring in stylet deployment direction (60).

FIG. 17 depicts an everting embodiment of the shield (5) including hub (105) and non-mesh sleeve (120).

FIG. 18 depicts representative eversion and fabrication features of the everting embodiment, including hub pull direction (110), eversion area (115), non-mesh sleeve (120), and sleeve eversion direction (125).

FIG. 19 depicts the everting shield advancing through tissue and performing progressive deployment and blunt dissection.

FIG. 20A depicts a surgical access shield (5) having a lateral side (135) and a medial side (130).

FIG. 20B depicts the surgical access shield (5) including a medial side coating (140).

FIG. 20C is a cross-sectional view taken along line A-A of FIG. 20A illustrating that the medial side wall thickness (T_{M}) is greater than the lateral side wall thickness (T_{L}).

FIG. 21A depicts preferential radial expansion (145) of the lateral side (135) of the shield (5) away from the medial side (130) during deployment with delivery stylet (75).

FIG. 21B is a cross-sectional view taken along line B-B of FIG. 21A further illustrating the preferential radial expansion (145).

FIG. 22A depicts an alternative embodiment of the surgical access shield (5) having higher mesh density on the medial side (130).

FIGS. 22B and 22C are cross-sectional views showing medial mesh density (D_{M}) greater than lateral mesh density (D_{L}).

### DETAILED DESCRIPTION OF EXAMPLE EMBODIMENTS

Reference is made herein to specific example embodiments, some of which are illustrated in the accompanying figures. While the subject matter is described in conjunction with these example embodiments, it will be understood that the disclosure is not limited to the illustrated embodiments. To the contrary, it is intended that various alternatives, modifications, and equivalents be encompassed within the scope of the claims as would be apparent to persons of skill in the art.

As used herein, the phrase transorbital surgical corridor refers to a pathway extending from an orbital incision (85) to a selected surgical target through orbital tissue and, where appropriate, through bone that has been opened, thinned, or removed. The phrase instrument workspace lumen refers to the protected internal working passage defined by the shield (5) and sized to receive one or more operative instruments, such as an endoscope, dissector, suction instrument, bipolar instrument, grasper, or drill accessory. In many illustrated embodiments that passage is the lumenal through path or lumen (26). The phrase medial orbital anatomical structures refers to tissue and neurovascular structures for which preferential protection is desired, including, by way of non-limiting example, the optic nerve, structures of the extraocular muscle cone, ophthalmic vasculature, sensory nerves, and associated soft tissues located along the medial side of the operative corridor.

The disclosed transorbital surgical access system includes an adaptive surgical access shield configured to navigate the confined anatomy of the orbit while maintaining a protected instrument path. In many of the illustrated embodiments, the surgical access shield is embodied as a braided or mesh-based shield (5), although other embodiments may be fenestrated, stent-like, or otherwise resilient and expandable. In use, the shield (5) is inserted in a low-profile state and then allowed or caused to expand within the corridor to create the lumen (26). The wall (24) of the shield (5) is positioned between the lumen (26) and medial orbital anatomical structures so that the wall (24) functions as a mechanical protection boundary during at least a substantial portion of the procedure. In this context, the mechanical protection boundary is provided by the physical barrier formed by the wall (24), which separates the instrument workspace lumen (26) from adjacent orbital tissues and limits direct contact between surgical instruments within the lumen (26) and medial orbital anatomical structures.

In a representative embodiment, the shield (5) is formed from a tubular braid (22) of interwoven filaments. Suitable filaments include superelastic or resilient metallic filaments, such as nitinol, stainless steel, cobalt-chromium alloys, or combinations thereof; high-strength polymeric filaments, such as polyethylene terephthalate, polyether ether ketone, polyamide, ultra-high molecular weight polyethylene, or combinations thereof; and composite filaments that combine metallic and polymeric components. Representative filament diameters may range from about 0.05 mm to about 0.25 mm. In representative expanded embodiments, the braid angle in the expanded configuration may be selected within a range of approximately 30 degrees to approximately 60 degrees to balance radial support, flexibility, axial foreshortening, and the desired expansion ratio.

The shield (5) may be configured to transform reversibly between a low-profile insertion configuration and an expanded operative configuration. In some embodiments, the low-profile insertion configuration has an inside diameter of approximately 2 mm to approximately 8 mm. When the shield (5) is released from axial tension or confinement, the shield (5) may foreshorten and radially expand to an expanded operative configuration having a larger working diameter, in some embodiments reaching a diameter of at least about 1.8 times the low-profile diameter and, in certain embodiments, up to about 3 times the low-profile diameter. The amount of expansion may be selected according to the anatomy to be traversed, the size of the instruments to be used, and the degree of tissue displacement considered acceptable for the intended procedure. In some embodiments, the shield (5) may be partially deployed, repositioned, reconstrained, and redeployed one or more times during a procedure.

Although a braided scaffold or tubular braid (22) is presently preferred in many embodiments, the shield (5) may also be formed as a fenestrated or stent-like body. For example, the shield (5) may be cut from a metallic tube, such as a nitinol hypotube, or from a resilient polymeric tube using laser cutting, etching, machining, molding, or additive manufacturing to create openings and struts that permit controlled radial expansion and axial flexibility. A polymeric sleeve, elastomeric coating, or thin film may partially or fully cover the openings of such a fenestrated body to provide a smoother tissue-contacting surface and to create a more continuous barrier where desired.

In some embodiments, the scaffold or braid (22) is coated or sleeved with an elastomeric material, such as medical-grade silicone, polyurethane, a fluoropolymer layer or fluoropolymer-containing coating, a hydrophilic coating, or combinations thereof. The coating may extend over inner surfaces, outer surfaces, or both, and may partially or completely bridge interstitial openings of the braid (22). In representative embodiments, the coating adds only minimal wall thickness while making the wall (24) substantially fluid-impermeable and reducing snagging of tissue or instruments. Lubricious surface treatments may also be applied to reduce friction during insertion, repositioning, instrument exchange, and removal. In some embodiments, one or more portions of the shield (5), coating, orientation indicium, or associated delivery components may include radiopaque, echogenic, or optically contrasting features to facilitate visualization during placement.

Preferably the combination of coating and braid will possess inherent lubricity or low frictional drag relative to instruments placed through and moved relative to the shield. Alternate coatings such as HDPE or Nylon would be candidates for the coating.

A principal feature of many embodiments is directional compliance. Directional compliance may be provided circumferentially, longitudinally, or both. The wall (24) of the shield (5) may include a first wall region configured to exhibit lower radial expandability and a second wall region configured to exhibit greater radial expandability. The lower-expandability region may be achieved, for example, by increasing filament density, reducing open area, increasing coating thickness, increasing coating durometer, using a tighter braid pitch, selecting stiffer constituent materials, increasing local wall thickness in a fenestrated embodiment, or combining two or more of those measures. Conversely, the greater-expandability region may be made more compliant by reducing filament density, increasing open area, decreasing coating thickness, using a lower-durometer coating, using a looser braid pitch, selecting more compliant constituent materials, or combining those measures.

In use, the lower-expandability wall region may be configured to be positioned adjacent medial orbital anatomical structures and the greater-expandability wall region may be configured to be positioned toward the lateral side of the corridor or toward the intended surgical workspace. As a result, when the shield (5) is displaced by instruments or by surrounding tissue, expansion occurs preferentially away from the more vulnerable medial anatomy. In representative embodiments, the first wall region therefore serves as a medial protective wall region and the second wall region serves as a laterally expandable working wall region. The difference between the two regions may be continuous or stepwise and may extend along the full length of the shield (5) or only along selected segments, such as the distal section (10), the mid section (15), or both.

To facilitate repeatable placement of asymmetric embodiments, the shield (5) may include an orientation indicium at or near its proximal portion. The indicium may be visual, tactile, radiopaque, color-based, embossed, notched, or otherwise detectable by the surgeon and may be aligned with the lower-expandability wall region. In some embodiments, the orientation indicium directly identifies first wall region of wall (24) having reduced radial expandability so that the surgeon can rotationally align the surgical access shield (5) with protected medial orbital anatomical structures prior to or during deployment, thereby consistently positioning the protective wall region between the instrument workspace lumen (26) and medial orbital anatomical structures. In guide-assisted embodiments, the mating relationship between the shield (5) and the protection guide (90) may also enforce or maintain a selected angular orientation during advancement.

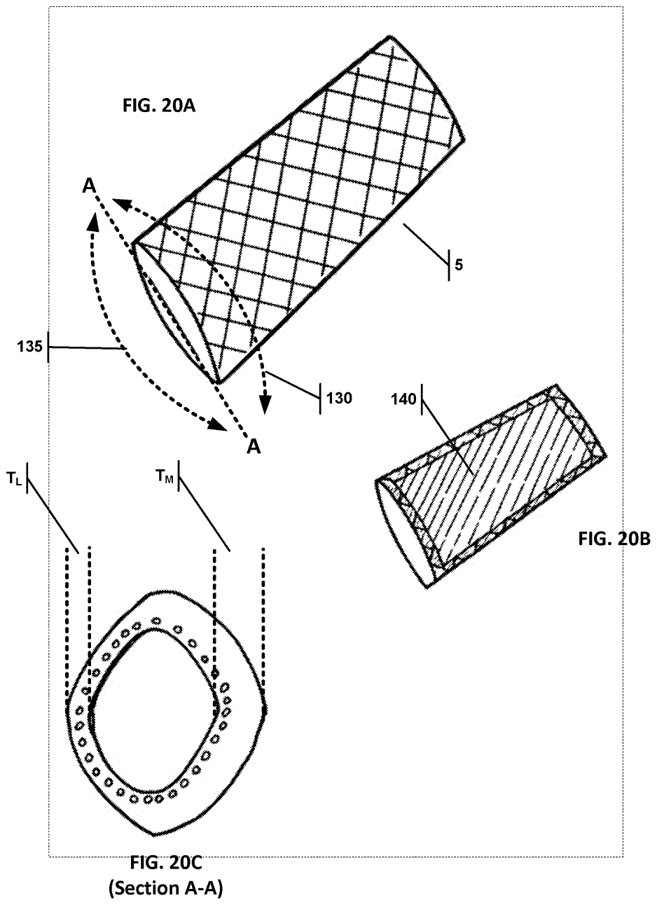

With reference to FIGS. 20A through 22C, further embodiments achieve the desired preferential radial expansion through specific structural asymmetries. As shown in FIGS. 20A and 20B, the surgical access shield (5) includes a distinct lateral side (135) and medial side (130). In the embodiment of FIG. 20C (cross-section A-A of FIG. 20A), the medial side wall thickness (T_{M}) is intentionally greater than the lateral side wall thickness (T_{L}). This differential thickness inherently restricts radial expansion on the medial side while permitting greater outward displacement on the lateral side. The medial side (130) may further include a medial side coating (140), as illustrated in FIG. 20B. The medial side coating (140) may be impregnated with pharmacologic compositions or may incorporate binding agents that further stiffen the medial wall, thereby reinforcing the mechanical protection boundary against delicate medial orbital anatomical structures.

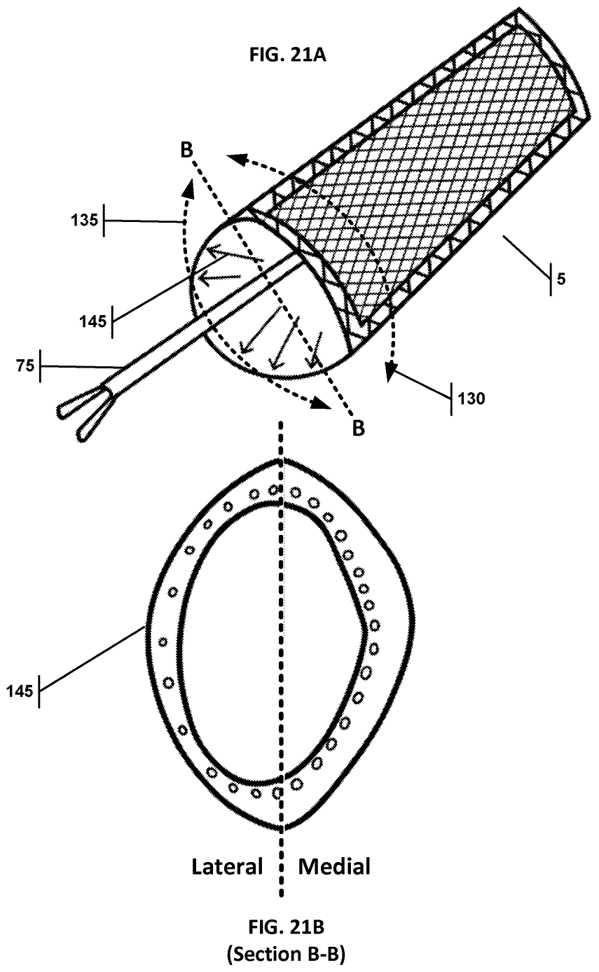

FIGS. 21A and 21B illustrate the resulting preferential radial expansion (145). After the shield (5) is positioned by delivery stylet (75) and released, expansion occurs preferentially on the lateral side (135) away from the medial side (130) and the protected orbital anatomy. The first wall region therefore serves as a medial protective wall region while the second wall region serves as a laterally expandable working wall region. This differential expansion may be continuous or stepwise and may extend along the full length of the shield or only along selected segments such as the distal section (10) or mid-section (15).

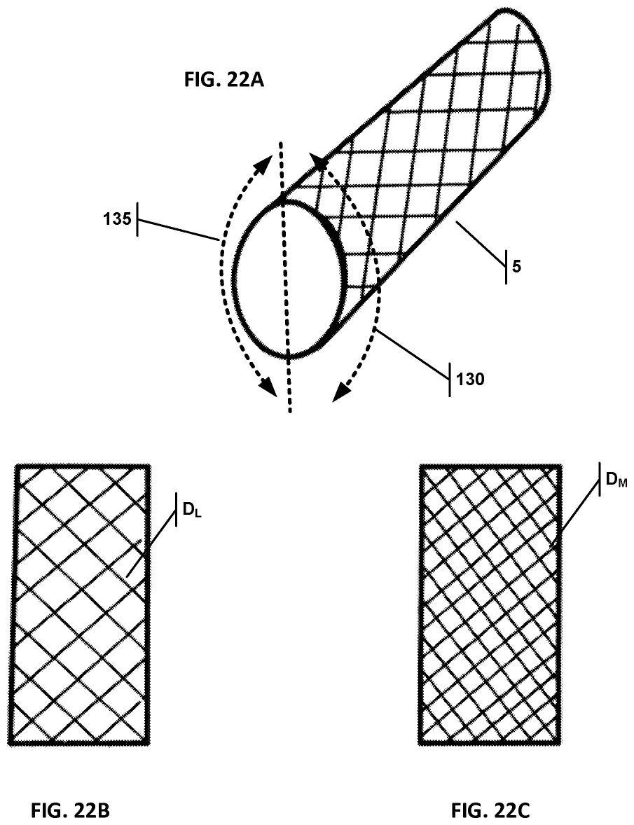

FIGS. 22A through 22C depict an alternative configuration for achieving preferential expansion. In this embodiment the medial side (130) of the braided scaffold possesses a higher mesh or filament density (D_M) than the lateral side (135) (D_{M}>D_{L}). The increased filament density on the medial side further limits radial compliance in the direction of protected medial orbital anatomical structures while the lower-density lateral side expands more readily into the surgical workspace.

### REFERENCE NUMERALS

5 surgical access shield
10 distal section
15 mid-section (waist)
20 proximal section
22 tubular braid
24 wall
26 instrument workspace lumen (lumenal through-path)
28 distal cone
30 proximal cone
32 confinement housing
34 plunger
35 plunger deployment direction
40 plunger removal direction
45 stylet
50 distal confinement structure
55 proximal confinement structure
60 stylet deployment direction
65 stylet retraction direction
70 distal eyelet (stylet receiver)
75 delivery stylet
80 distal stop
85 incision
90 protection guide
95 receiver slot
100 receiver slot interface
105 hub
110 hub pull direction
115 eversion area
120 non-mesh sleeve
125 sleeve eversion direction
130 medial side
135 lateral side
140 medial side coating
145 preferential radial expansion

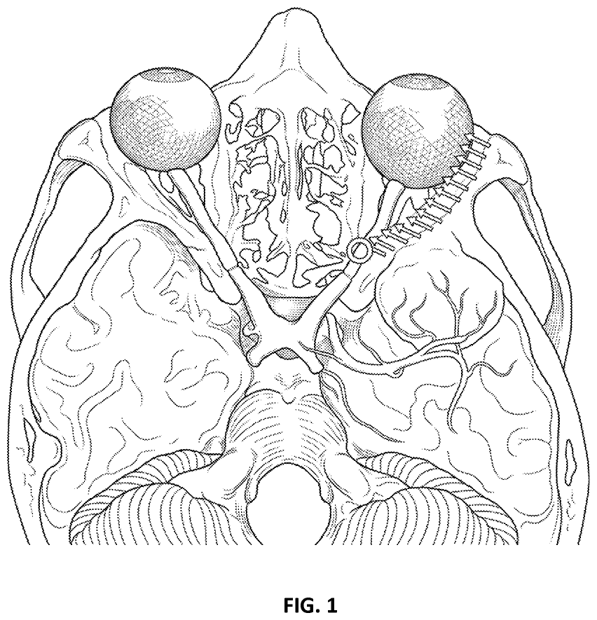

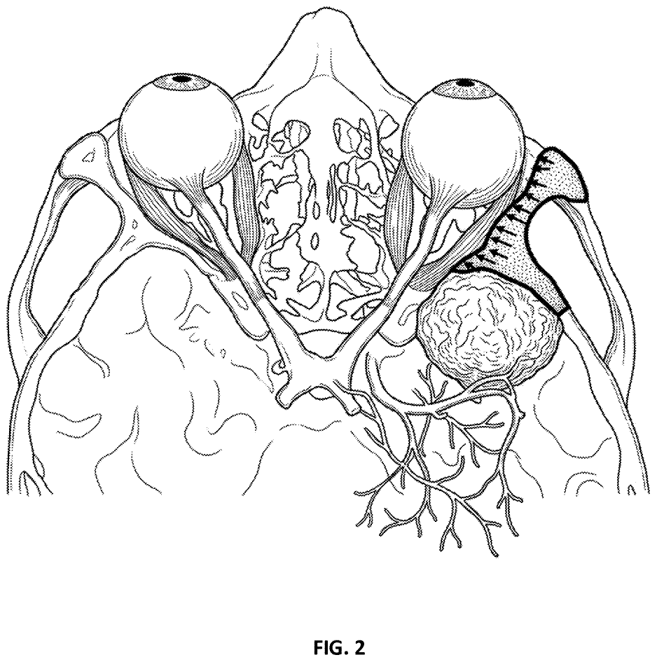

With reference to FIGS. 1 and 2, the transorbital surgical corridor may be formed lateral to the orbital muscle cone while delicate medial orbital anatomical structures may remain vulnerable to inadvertent instrument passage or excessive outward displacement. Those figures illustrate the need for a device that creates a defined operative path while preserving a protective medial boundary. The shield (5) addresses that need by occupying the corridor and interposing the wall (24) between operative instruments and the protected anatomy.

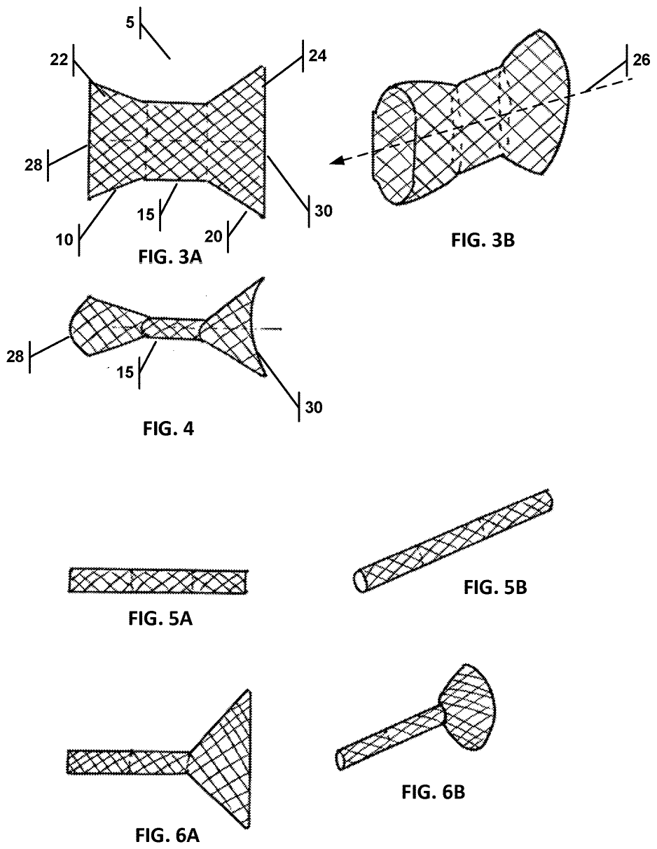

With reference to FIGS. 3A, 3B, and 4, one illustrative embodiment of the shield (5) has an hourglass shape. The shield (5) includes distal section (10), midsection or waist (15), and proximal section (20). In the illustrated embodiment, the shield (5) is formed from a continuous tubular braid (22) defining wall (24) and instrument workspace lumen (26). Preferably, the braid (22) is coated or sleeved in a polymer to cover the interstices and make the wall (24) less permeable to fluid and, in some embodiments, substantially fluid-impermeable or gas-impermeable. The distal section (10) may define a distal cone (28), and the proximal section (20) may define a proximal cone (30).

The distal cone (28) may have an oblong, circular, elliptical, or otherwise non-circular shape and may help maintain the surgical field by tenting tissue and allowing less restricted triangulation of elongated surgical instruments. The midsection (15) may be waist-like to provide a pathway conduit that minimizes pressure on the surgical pathway while still offering a tissue-compliant conduit and an expandable operative space for instruments. The proximal cone (30) is preferably extracorporeal or positioned at the entry region of the surgical pathway and may provide a funnel-like opening for easier insertion of instruments. The proximal cone (30) may also cover or shield tissue at the incision (85), including adjacent tissue such as the globe, from unintentional instrument contact.

FIGS. 5A and 5B show the shield (5) in a drawn-down low-profile configuration. In that state, the shield (5) is generally tubular and the distal and proximal funnel portions are compacted in line with the reduced-profile central portion. This configuration is a representative insertion or delivery profile. The low-profile state may be established by confinement within a housing, by axial elongation over a stylet, by traction applied to the shield itself, or by other mechanical means.

FIGS. 6A and 6B illustrate alternate configurations. In FIG. 6A, the proximal cone (30) remains expanded while a more distal portion remains drawn down. In FIG. 6B, the proximal cone (30) is again present, but the distal cone (28) is omitted. In such embodiments, the reduced-profile section may nevertheless maintain the ability to expand spontaneously or in response to instrument loading to accommodate instruments and the articulation or manipulation of instruments, while defaulting toward a lower-profile condition in the absence of instrument loading.

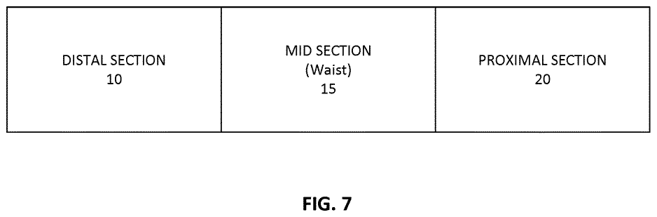

FIG. 7 schematically divides the shield (5) into distal section (10), midsection (15), and proximal section (20). The distal section (10) may draw down to a low profile for insertion and may then expand into a circular or oblong distal cone (28) to tent or gently displace tissue adjacent the surgical site. In some embodiments, the distal section (10) need not form a pronounced cone, but may instead open, yield, or flare in response to the urging of instruments. The midsection (15) may draw down to a low profile and may remain relatively low profile as an instrument-compliant virtual-space conduit, while optionally expanding to open or enlarge the pathway. The proximal section (20) may expand to provide a larger-profile insertion guide path and may also shield the incision site or adjacent tissue during placement and removal of instruments.

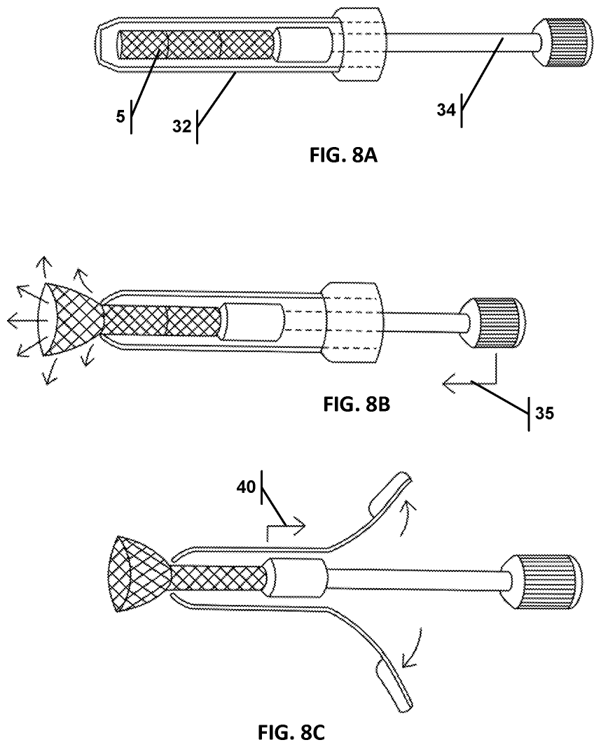

FIGS. 8A-8C depict a delivery system in which confinement housing (32) confines the shield (5) in a low profile. A plunger (34) may push in plunger deployment direction (35) to deploy the mesh shield (5) as the shield exits the confinement housing (32), as shown in FIG. 8B. Upon movement in plunger removal direction (40), the confinement housing (32) may optionally split, peel away, or otherwise open. Such a splitable housing is particularly useful when the proximal end of the shield (5) needs to remain expanded, or cannot readily be compressed to fit within a closed housing lumen.

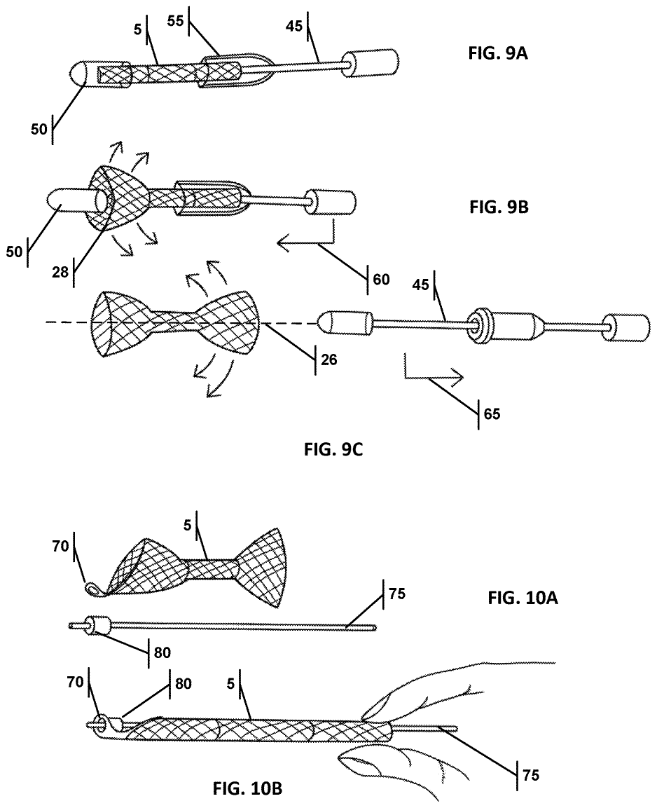

FIGS. 9A-9C depict a deployment system in which the shield (5) is delivered over stylet (45) with distal confinement structure (50) and proximal confinement structure (55), each illustrated as a cup-like member. The stylet (45) is preferably sufficiently rigid to resist buckling under the radial force load of the contracted shield (5) and the resistance encountered during insertion into the surgical pathway. The confinement structures (50, 55) constrain the distal section (10) and proximal section (20) of the shield (5), while axial stretch of the shield (5) over the stylet (45) contracts the shield to a lower profile.

In one representative sequence, the elongated, confined, and stretched assembly is inserted through the surgical pathway in the configuration shown in FIG. 9A. The stylet (45) is then advanced distally relative to the shield (5) in stylet deployment direction (60), allowing the distal confinement structure (50) to move free of the confined distal section (10). The distal section (10) of the shield (5) then expands into distal cone (28), as shown in FIG. 9B. Thereafter, the stylet (45) is retracted in stylet retraction direction (65), so that the distal confinement structure (50) is withdrawn through lumen (26) while the proximal confinement structure (55) is withdrawn from proximal section (20), thereby allowing formation of proximal cone (30). The stylet (45) with the confinement structures (50, 55) may then be fully removed from the shield (5) and the surgical field, as shown in FIG. 9C.

FIGS. 10A and 10B depict an embodiment in which the shield (5) includes distal eyelet or stylet receiver (70) and cooperates with delivery stylet (75). In this embodiment, the shield (5) is loaded over the delivery stylet (75) as shown in FIG. 10B, and the delivery stylet (75) includes distal stop (80) that engages distal eyelet (70). The user or an associated actuator mechanism may apply proximal traction to elongate the shield (5), thereby causing the shield (5) to elongate and constrict over the delivery stylet (75). The elongated assembly is inserted through the surgical pathway, after which the proximal end of the shield (5) may be held fixed by the user, the pathway, or an actuator mechanism while the delivery stylet (75) is removed. Proximal restraint may then be released, allowing the shield (5) to spontaneously expand into its expanded operative configuration.

A reversible, selectively expandable and constrictable embodiment may be made by permanently bonding distal stop (80) of the delivery stylet (75) to distal eyelet (70). Relative distal or proximal motion of the delivery stylet (75) would then cause corresponding contraction or expansion of the shield (5). Such an arrangement may be useful when the user wishes to deploy the shield (5) progressively along the surgical path, deploy the shield during creation of the surgical path, reposition the shield in a low-profile state, or remove the shield in a low-profile state.

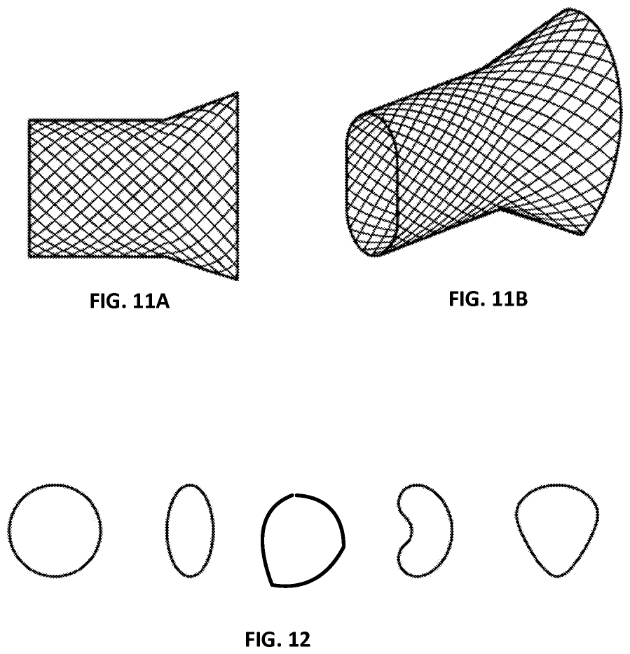

FIGS. 11A and 11B depict embodiments of the shield (5) without a drawn-down waist-like mid-section (15). In such embodiments, the shield (5) may still define a lumen (26), provide a protective wall (24), and include a proximal flare or cone (30), while using a more gradually tapered or substantially untapered profile between the distal and proximal ends. FIG. 12 depicts representative non-limiting cross-sectional profiles for the shield (5), including circular, elliptical, lobed, bean-shaped, and tear-drop profiles. Such profiles may be selected according to the anatomy of the corridor, the side of preferential expansion, or the intended instruments.

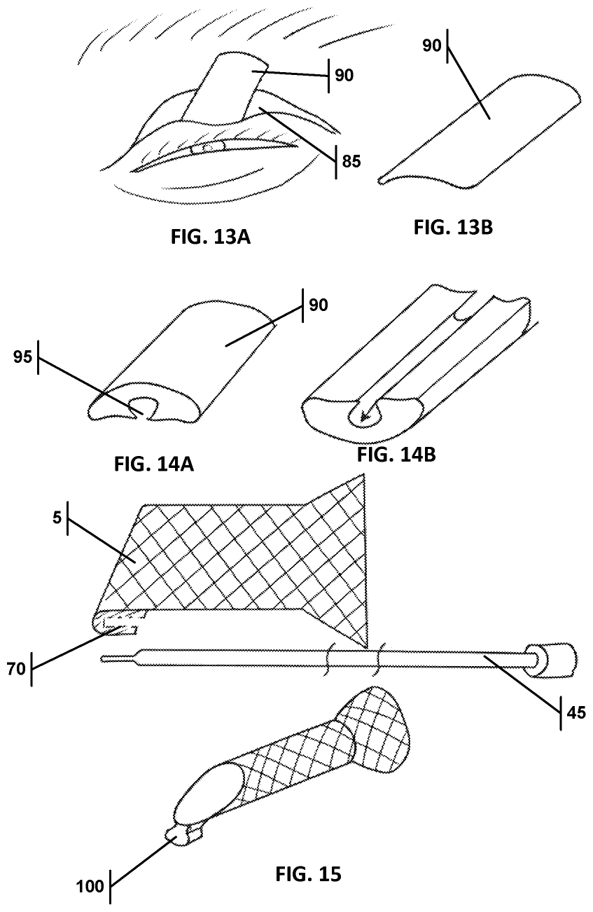

FIGS. 13A and 13B depict a protection guide (90) inserted through incision (85) and into the surgical pathway. The protection guide (90) may be thin and have sufficient rigidity not to buckle during placement, while also allowing tissue retraction and manipulation. The protection guide (90) may protect orbital anatomy from inadvertent damage from instrument passage and may establish, maintain, preserve, or enlarge an initial pathway for subsequent placement of the shield (5).

FIGS. 14A and 14B depict the protection guide (90) with receiver slot (95). The receiver slot (95) acts as a guide for slidable passage of a shield configured with a mating mechanical feature, shown in FIG. 15 as receiver slot interface (100). The protection guide (90) and shield (5) may be used in conjunction in various ways. The protection guide (90) may be fully placed first and the shield (5) then advanced along the protection guide (90). Alternatively, the protection guide (90) and shield (5) may be jointly advanced, or may be placed in an alternating and stepwise manner in which the protection guide (90) advances, then the shield (5) advances, then the protection guide (90) re-advances, and then the shield (5) advances further. The protection guide (90) may remain in place throughout the procedure or may be removed after the shield (5) is placed. In asymmetric embodiments, the receiver slot (95) and receiver slot interface (100) may also assist in maintaining a selected angular orientation of the shield (5) during advancement.

FIG. 15 depicts the shield (5) with receiver slot interface (100) and stylet (45). In the illustrated embodiment, the shield (5) is loaded onto stylet (45) at distal eyelet or stylet receiver (70) located at or near the receiver slot interface (100). The shield (5) may be stretched to a low profile adjacent to stylet (45), and the receiver slot interface (100) may then be engaged in receiver slot (95) of protection guide (90).

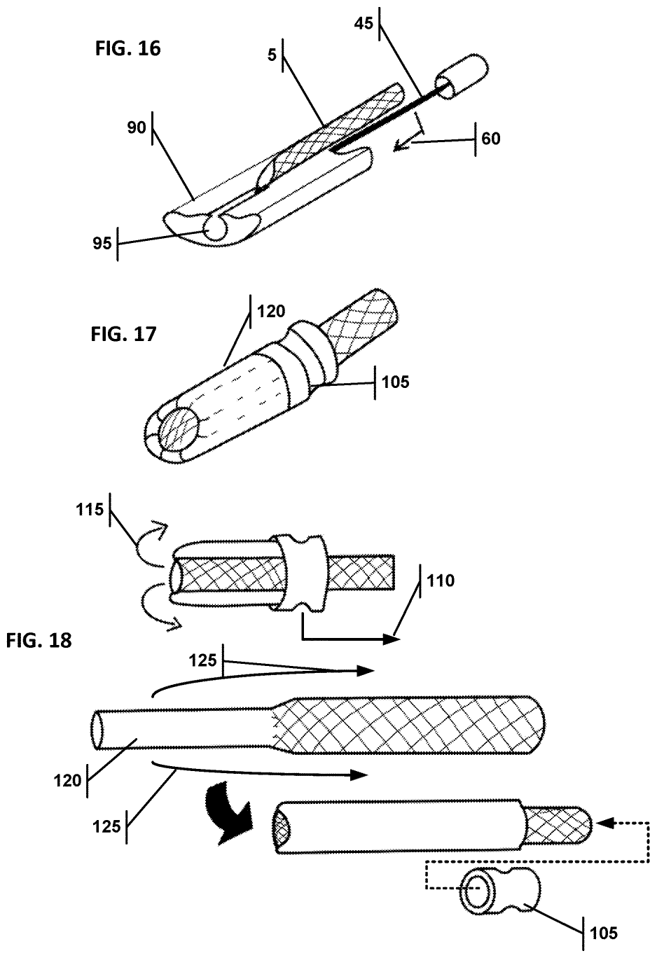

As shown in FIG. 16, the shield (5) and stylet (45) may be pushed distally along protection guide (90), with advancement occurring generally in stylet deployment direction (60). When the shield (5) and stylet (45) reach the distal end of receiver slot (95), the shield (5) may disengage by exiting the slot distally. The protection guide (90) and stylet (45) can then be removed, leaving the shield (5) in place for spontaneous expansion at the intended operative location, with the accessory guide and stylet removed from the surgical field. Additionally, the stylet (45) may be permanently attached to distal section (10) of the shield (5) so that the surgeon may selectively use the stylet (45) to expand or contract the shield profile.

FIG. 17 depicts a version of the shield (5) that is adapted to evert from the proximal side of the surgical pathway in a conveyor-like manner. The purpose of this concept is to deploy the shield (5) progressively with an extruding forward motion so that the user may deploy the shield (5) progressively and atraumatically. The everting shield may advance from proximal to distal through the incision (85), may help define or augment the incision (85), and may provide blunt dissection. In the illustrated embodiment, the shield (5) is associated with hub (105) and non-mesh sleeve (120).

In representative everting embodiments, the shield (5) may be made from a continuous tubular structure in which a portion is everted backward and redundant over a non-everted core of mesh or other scaffold. Hub (105) may be attached to the most proximal end of the initially everted portion so that the user can grasp the hub (105) and pull in hub pull direction (110) to induce eversion at eversion area (115) and advancement as desired. In one form, the initially everted portion is non-mesh sleeve (120) that is integral with the more distal mesh-based portion of the shield (5). Use of non-mesh sleeve (120) may reduce compounding of wall thickness with a double layer of mesh and may allow, when fully everted, a single-layer shield to remain in place.

FIG. 18 depicts one representative fabrication approach for an everting sleeve or shield. The mesh portion may be positioned on an elongated mandrel, after which a polymer may coat the mesh and an additional length beyond the mesh to form non-mesh sleeve (120). When the mandrel is removed, the resulting structure may have non-mesh sleeve (120) integral with the polymer coating the mesh. Non-mesh sleeve (120) may then be everted over the mesh in sleeve eversion direction (125), thereby creating a dual-layer manufactured state in which the sleeve is available for the user to grasp and pull via hub (105) to induce eversion at the time of surgical placement. Hub (105) may be added to the initially everted sleeve during manufacture or in a subsequent assembly step.

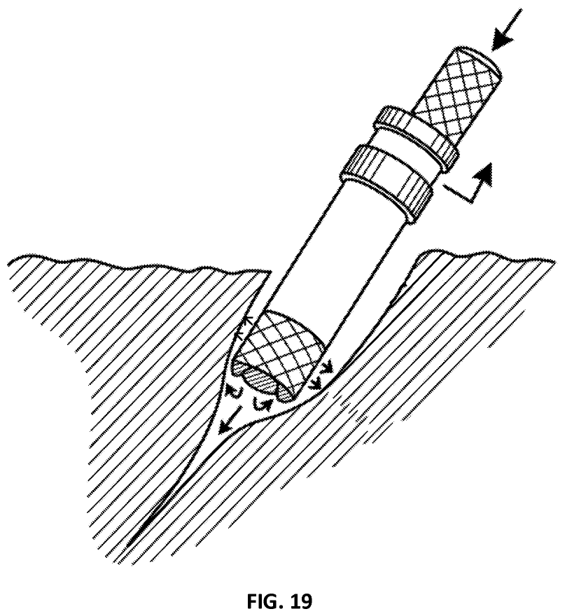

FIG. 19 depicts the everting shield everting, dissecting, and advancing through tissue at an incision. During such use, the leading portion of the shield (5) may present a smooth outer surface that gently separates tissue planes while the shield continues to define a protected lumen for subsequent instrument passage.

The proximal funnel or cone (30) may remain at or adjacent the incision (85) during use and may thereby perform several functions. The proximal cone (30) may protect the globe or peri-incisional soft tissues from instrument contact, facilitate entry of instruments into lumen (26), provide an abutment or abutment surface that helps resist distal migration of the shield (5), and reduce unintended rotation of the shield (5) by increasing the area of contact at the entry region. Surface texturing, local compliance changes, tabs, flanges, or other retention features may also be provided to improve positional stability, provided that such features do not interfere with atraumatic introduction or removal.

The openings of the shield (5) may include atraumatic rims. In braided embodiments, end portions of braid (22) may be folded, rolled, tucked, bonded, heat-set, or covered with a coating layer to reduce fraying and minimize tissue snagging. Preferably the combination of coating and braid will possess inherent lubricity or low frictional drag relative to instruments placed through and moved relative to the shield. Alternate coatings such as HDPE or Nylon would be candidates for the coating.

In fenestrated embodiments, cut edges may be polished, rounded, overmolded, covered by a sleeve, or otherwise treated to provide a smooth edge profile. The inner surface of the shield (5) may remain at least partially supported by the scaffold architecture even when coated, thereby maintaining sufficient open internal space for instrument exchange while limiting drag.

In use, the surgeon creates a transorbital surgical corridor through a superior eyelid crease incision, a lateral orbital approach, or another suitable orbital access route. The corridor may be created using conventional dissection and, where appropriate, with limited bone removal. Protection guide (90) may then be inserted to establish or maintain the pathway and to retract or protect adjacent tissue. The shield (5), in a low-profile state, is advanced into the corridor either directly, over stylet (45), over delivery stylet (75), through confinement housing (32), along protection guide (90), or by everting deployment.

Before or during insertion of an asymmetric embodiment, the surgeon may use the proximal orientation indicium to align the lower-expandability wall region of wall (24) with the medial side of the orbit, thereby rotationally orienting the surgical access shield (5) so that the protective wall region is positioned between lumen (26) and medial orbital anatomical structures. The shield (5) is then released or otherwise allowed to assume its operative configuration. After deployment, wall (24) of the shield (5) is positioned between lumen (26) and the medial orbital anatomical structures, thereby establishing the intended mechanical protection boundary. Operative instruments are introduced through proximal cone (30) and advanced through lumen (26) toward the target. As the instruments are manipulated, the more compliant wall region may locally expand to accommodate movement while the more protective wall region continues to shield the medial anatomy.

Midsection (15), when present, may expand and contract reversibly in response to instrument passage and manipulation. The distal opening of distal section (10) may also enlarge transiently during contact with instruments and then recover elastically when the contact load is reduced. At the conclusion of the procedure, the shield (5) may be returned to a lower-profile state by withdrawing instruments, applying axial tension, drawing the shield back over stylet (45) or delivery stylet (75), retracting the everted layer, or otherwise reversing the selected deployment mechanism. The shield (5) is then removed through incision (85) with reduced trauma to the access pathway.

The disclosed system therefore provides a transorbital access structure that is both protective and adaptive. By combining instrument workspace lumen (26), the mechanical protection boundary provided by wall (24), optional directional compliance, and multiple placement options, the system can improve safety, visualization, and instrument maneuverability in transorbital procedures. In some embodiments, the shield (5) and associated delivery components may be provided individually or as part of a sterile procedural kit. The particular dimensions, materials, braid parameters, coating characteristics, and deployment details described herein are illustrative and may be varied to suit the intended anatomy and procedure without departing from the scope of the claims.

While the disclosure has been described with reference to representative embodiments, persons of ordinary skill in the art will recognize that additions, deletions, substitutions, and modifications may be made without departing from the scope of the invention. The drawings and description should therefore be regarded as illustrative rather than limiting.

1. A surgical access system for transorbital surgery, comprising:
a surgical access shield (5) configured to be positioned within a transorbital surgical corridor extending between an orbital incision (85) and a surgical target;
the surgical access shield (5) defining an instrument workspace lumen (26) and comprising a wall (24) that, when the surgical access shield (5) is positioned within the transorbital surgical corridor, is disposed between the instrument workspace lumen (26) and medial orbital anatomical structures to establish a mechanical protection boundary.

2. The surgical access system of claim 1, wherein the wall (24) comprises a first wall region having a lower radial expandability than a second wall region, the first wall region being configured to be positioned adjacent protected orbital structures and the second wall region being configured to permit greater expansion toward a surgical workspace.

3. The surgical access system of claim 2, wherein the first wall region is configured for medial placement and the second wall region is configured for lateral placement such that the surgical access shield (5) expands preferentially toward a lateral side of the surgical corridor.

4. The surgical access system of claim 1, wherein the surgical access shield (5) comprises a tubular braid (22) formed from interwoven filaments arranged in a helical braid pattern.

5. The surgical access system of claim 4, wherein the tubular braid (22) is coated or sleeved with an elastomeric material that fills at least a portion of interstitial spaces of the braid to form a substantially fluid-impermeable barrier at the wall (24).

6. The surgical access system of claim 1, wherein the surgical access shield (5) has a deployed hourglass shape including a distal section (10), a mid-section or waist (15) having a smaller cross-section than the distal section (10), and a proximal section (20) defining a proximal cone or funnel (30).

7. The surgical access system of claim 6, wherein the distal section (10) defines a distal cone (28) and the proximal cone or funnel (30) has a characteristic dimension that is about 1.5 to about 3.0 times greater than a corresponding dimension of the distal section (10).

8. The surgical access system of claim 1, further comprising a protection guide (90) having a receiver slot (95), wherein the surgical access shield (5) includes a receiver slot interface (100) configured to be slidably received in the receiver slot (95) for guided placement within the transorbital surgical corridor.

9. The surgical access system of claim 1, wherein the surgical access shield (5) is configured to be constrained in a low-profile insertion configuration within a confinement housing (32) and to expand upon release from the confinement housing (32).

10. (canceled)

11. (canceled)

12. (canceled)

13. (canceled)

14. (canceled)

15. (canceled)

16. (canceled)

17. (canceled)

18. (canceled)

19. The surgical access system of claim 1, wherein the surgical access shield (5) has a circular, elliptical, lobed, bean-shaped, or tear-drop cross-sectional profile.

20. A method of performing a transorbital surgical procedure, comprising:
creating a transorbital surgical corridor through orbital tissue;
advancing a surgical access shield (5) into the transorbital surgical corridor;
positioning a wall (24) of the surgical access shield (5) between an instrument workspace lumen (26) and medial orbital anatomical structures; and
expanding the surgical access shield (5) to establish the lumen (26) while maintaining a mechanical protection boundary between one or more surgical instruments and the medial orbital anatomical structures.

21. The method of claim 20, wherein advancing the surgical access shield (5) comprises constraining the surgical access shield (5) in a low-profile state within a confinement housing (32) and releasing the surgical access shield (5) from the confinement housing (32) by plunger (34) advancement, housing retraction, or both.

22. The method of claim 20, wherein advancing the surgical access shield (5) comprises stretching the surgical access shield (5) over a stylet (45) into a low-profile insertion configuration and releasing the surgical access shield (5) by relative movement between the stylet (45) and the surgical access shield (5).

23. (canceled)

24. (canceled)

25. (canceled)

26. The method of claim 20, wherein the surgical access shield (5) is deployed progressively by everting a non-mesh sleeve (120) and applying traction to hub (105) in hub pull direction (110) to create eversion at eversion area (115).

27. The method of claim 20, wherein a distal opening of distal section (10) expands spontaneously and reversibly in response to manipulation of one or more surgical instruments.

28. The method of claim 20, further comprising adapting the surgical access shield (5) to a low-profile final state before removing the surgical access shield (5) from orbital tissue.

29. The method of claim 20, wherein the surgical access shield (5) comprises a tissue-conformal mid-section (15) that expands or contracts reversibly in response to instrument manipulations within the lumen (26).

30. The surgical access system of claim 2, wherein the first wall region has a greater wall thickness than the second wall region.

31. The surgical access system of claim 30, wherein the first wall region comprises the medial side (130) and the second wall region comprises the lateral side (135) of the surgical access shield (5).

32. The surgical access system of claim 2, wherein the first wall region further comprises a medial side coating (140) configured to restrict radial expansion.

33. The surgical access system of claim 32, wherein the medial side coating (140) is impregnated with a pharmacologic composition or includes binding agents that increase stiffness of the medial side.

34. The surgical access system of claim 2, wherein the first wall region has a higher filament density than the second wall region.

35. The surgical access system of claim 34, wherein the higher filament density is present on the medial side (130) of the surgical access shield (5).

36. The surgical access system of claim 2, wherein preferential radial expansion (145) occurs on the lateral side (135) away from the medial side (130) upon deployment of the surgical access shield (5).
